(19) United States
(12) Patent Application Publication
(10) Pub. No.: US 2021/0321878 A1
(43) Pub. Date: Oct. 21, 2021

(54) HEALTH MANAGEMENT SYSTEM AND HEALTH MANAGEMENT METHOD

(71) Applicant: INTERNATIONAL MOBILE IOT CORP., TAIPEI CITY (TW)

(72) Inventor: Chia Hwa Lee, TAIPEI CITY (TW)

(73) Assignee: INTERNATIONAL MOBILE IOT CORP., TAIPEI CITY (TW)

(21) Appl. No.: 17/024,729

(22) Filed: Sep. 18, 2020

Related U.S. Application Data
(60) Provisional application No. 63/013,525, filed on Apr. 21, 2020.

(30) Foreign Application Priority Data
Aug. 7, 2020 (TW) 109126765

(51) Int. Cl. A61B 5/0205; G08B 21/18; H04L 29/08; A61B 5/00; A61B 5/11; A61B 5/0476; A61B 5/0488; A61B 7/00; A61B 5/1172

(52) U.S. Cl. CPC A61B 5/0205; A61B 5/02405; H04L 67/10; A61B 5/0022; A61B 5/6807; A61B 5/112; A61B 5/0476; A61B 5/0488; A61B 7/008; A61B 5/746; A61B 5/7275; A61B 5/4866; A61B 5/4064; A61B 5/681; A61B 5/6803; A61B 5/1112; A61B 5/1172; G08B 21/18

(57) ABSTRACT

A health management system and a health management method are provided. The health management method includes: measuring a position information of a person; determining to measure a physiological state information of the person according to the position information; and generating a physiological state report according to the physiological state information.

### CROSS-REFERENCE TO RELATED APPLICATION

This application claims the priority benefits of U.S. provisional application Ser. No. 63/013,525, filed on Apr. 21, 2020, and Taiwan application serial no. 109126765, filed on Aug. 7, 2020. The entirety of each of the above-mentioned patent applications is hereby incorporated by reference herein and made a part of this specification.

### BACKGROUND OF THE INVENTION

### Field of the Invention

The invention relates to a health management system and a health management method.

### Description of Related Art

At present, more and more people pay more attention to independent health management and disease prevention. In addition, as many countries become an aging society, the need for long-term care is gradually increasing. However, regardless of services such as health management or long-term care, professionals are needed. In this way, in addition to spending a lot of personnel costs, the privacy of service users may also be violated. Accordingly, how to achieve automated health management services is an object of those skilled in the art.

### SUMMARY OF THE INVENTION

The invention provides a health management system and a health management method that may monitor the physiological state of a person in a specific space.

A health management system of the invention is suitable for monitoring a physiological state of a person in a specific space, and includes a physiological state sensor, a positioning system, a local server, and a cloud server. The positioning system measures a position information of the person. The local server is communicatively connected to the positioning system of the physiological state sensor, wherein the local server determines to measure a physiological state information of the person using the physiological state sensor according to the position information. The cloud server is communicatively connected to the local server, wherein the cloud server generates a physiological state report according to the physiological state information.

In an embodiment of the invention, the health management system further includes a wearable device. The physiological state sensor is disposed at the wearable device to measure the physiological state information of the person wearing the wearable device.

In an embodiment of the invention, the wearable device includes a shoe, wherein the physiological state sensor includes a nine-axis sensor, and the physiological state information includes a gait information.

In an embodiment of the invention, the physiological state sensor includes a photoplethysmography sensor, wherein the physiological state information includes a heart rhythm variability and a blood pressure.

In an embodiment of the invention, the physiological state sensor includes an electroencephalography sensor, wherein the physiological state information includes an electroencephalogram.

In an embodiment of the invention, the physiological state sensor includes a thermometer, wherein the physiological state information includes a body temperature.

In an embodiment of the invention, the physiological state sensor includes an electromyography sensor, wherein the physiological state information includes an electromyogram.

In an embodiment of the invention, the physiological state sensor includes an electrode pad, wherein the physiological state information includes a bowel sound.

In an embodiment of the invention, the wearable device includes a shoe, wherein the physiological state sensor includes a neck-mounted device, and the physiological state information includes a displacement information.

In an embodiment of the invention, the cloud server generates the physiological state report including a warning message related to a brain disease risk or a metabolism deterioration risk according to the gait information.

In an embodiment of the invention, the gait information includes a stepping length and a step width, wherein the cloud server generates the physiological state report including a warning message related to a brain disease risk in response to the stepping length being less than a stepping length threshold or the step width being greater than a step width threshold.

In an embodiment of the invention, the cloud server generates the physiological state report including a warning message related to a brain disease risk or a cardiovascular disease risk in response to the blood pressure being greater than a blood pressure threshold.

In an embodiment of the invention, the cloud server generates the physiological state report including a warning message related to a brain disease risk, a cardiovascular disease risk, or a metabolism deterioration risk according to the heart rhythm variability.

In an embodiment of the invention, the cloud server generates the physiological state report including a warning message related to a brain disease risk according to the electroencephalogram.

In an embodiment of the invention, the cloud server generates the physiological state report including a warning message related to a cardiovascular disease risk in response to the body temperature being greater than a body temperature threshold.

In an embodiment of the invention, the cloud server generates the physiological state report including a warning message related to a brain disease risk according to the electromyogram.

In an embodiment of the invention, the cloud server generates the physiological state report including a warning message related to a metabolism deterioration risk according to the bowel sound.

In an embodiment of the invention, the cloud server generates the physiological state report including a warning message related to a metabolism deterioration risk according to the displacement information.

In an embodiment of the invention, the wearable device includes a smart bracelet, a head-mounted device, or a neck-mounted device.

In an embodiment of the invention, the wearable device further includes a head-mounted device.

In an embodiment of the invention, the wearable device includes a smart bracelet, a head-mounted device, or a neck-mounted device.

In an embodiment of the invention, the health management system further includes an environmental state sensor. The environmental state sensor is communicatively connected to the local server, wherein the local server measures an environmental state information using the environmental state sensor.

In an embodiment of the invention, the health management system further includes an air conditioning device. The air conditioning device is communicatively connected to the cloud server, wherein the environmental state sensor includes an air detector, the environmental state information includes an air quality, and the cloud server activates the air conditioning device according to the air quality.

In an embodiment of the invention, the health management system further includes a temperature adjustment device. The temperature adjustment device is communicatively connected to the cloud server, wherein the environmental state sensor includes an environmental thermometer, the environmental state information includes an environmental temperature, and the cloud server activates the temperature adjustment device according to the environmental temperature.

In an embodiment of the invention, the local server further determines to measure the physiological state information of the person using the physiological state sensor according to at least one of a time information, an environmental temperature, and an air quality.

In an embodiment of the invention, the local server determines a time that the person stays at a preset position in the specific space according to the position information and the time information, and measures the physiological state information of the person using the physiological state sensor corresponding to the preset position in response to the time being greater than a time threshold.

In an embodiment of the invention, the health management system further includes a wearable device, wherein the positioning system includes a first wireless transceiver, a second wireless transceiver, a third wireless transceiver, and a fourth wireless transceiver, and the first wireless transceiver is disposed at the wearable device; and the second wireless transceiver, the third wireless transceiver, and the fourth wireless transceiver are respectively disposed at different positions in the specific space.

In an embodiment of the invention, the positioning system transmits a signal via the first wireless transceiver, and receives the signal via the second wireless transceiver, the third wireless transceiver, and the fourth wireless transceiver to execute a triangulation positioning measurement to generate the position information.

In an embodiment of the invention, the positioning system transmits a second signal via the second wireless transceiver, transmits a third signal via the third wireless transceiver, transmits a fourth signal via the fourth wireless transceiver, and receives the second signal, the third signal, and the fourth signal via the first wireless transceiver to execute a triangulation positioning measurement to generate the position information.

In an embodiment of the invention, the positioning system prestores a magnetic fingerprint corresponding to the specific space, wherein the positioning system radiates a magnetic signal via the second wireless transceiver, receives the magnetic signal via the first wireless transceiver, and generates the position information according to the magnetic signal and the magnetic fingerprint received by the first wireless transceiver.

In an embodiment of the invention, the health management system further includes a wearable device, wherein the positioning system includes a nine-axis sensor disposed on the wearable device, the positioning system measures a movement information of the person wearing the wearable device via the nine-axis sensor, and generates the position information according to the movement information.

In an embodiment of the invention, the movement information includes at least one of the following: a current position, a speed, a heading, and a magnetic strength of a specific direction.

In an embodiment of the invention, the cloud server transmits the physiological state report to a terminal device sending an access request in response to receiving the access request.

A health management method of the invention is suitable for monitoring a physiological state of a person in a specific space, and includes: measuring a position information of the person; determining to measure a physiological state information of the person according to the position information; and generating a physiological state report according to the physiological state information.

Based on the above, in the invention, the user may be guided to understand the adverse effects of living habits without infringing on the privacy of the user, thereby preventing the occurrence of diseases (such as cardiovascular disease or brain disease).

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 shows a diagram of a health management system according to an embodiment of the invention.

FIG. 2 shows a diagram of a local server according to an embodiment of the invention.

FIG. 3 shows a diagram of a cloud server according to an embodiment of the invention.

FIG. 4 shows a diagram of a positioning system according to an embodiment of the invention.

FIG. 5 shows a diagram of a gait information according to an embodiment of the invention.

FIG. 6 shows a flowchart of a health management method according to an embodiment of the invention.

### DESCRIPTION OF THE EMBODIMENTS

The occurrence of cardiovascular disease or brain disease may be predicted by some early signs. Take cardiovascular disease as an example, when the water in the blood becomes less and the blood becomes thick, the blood vessels are readily blocked. Therefore, the water in the blood may be used as a leading indicator to predict cardiovascular disease. Moreover, long-term lack of good quality sleep may readily cause the β-amyloid produced by the brain nerve response to not be metabolized in time and gradually accumulate. The accumulated β-amyloid may hinder the transmission of cranial nerve signals and cause the decline or death of brain cells. After the hippocampal gyms is affected by β-amyloid to a certain extent, the hippocampal gyms does not recover. Therefore, the proportion of quality sleep may be used as a leading indicator for predicting brain disease. In addition, after the patient develops brain disease, the patient's speech may begin to slur (indirectly affecting the ability to chew), or the patient's gait may be changed. The brain waves of the damaged area of the brain nerve of the patient is also changed.

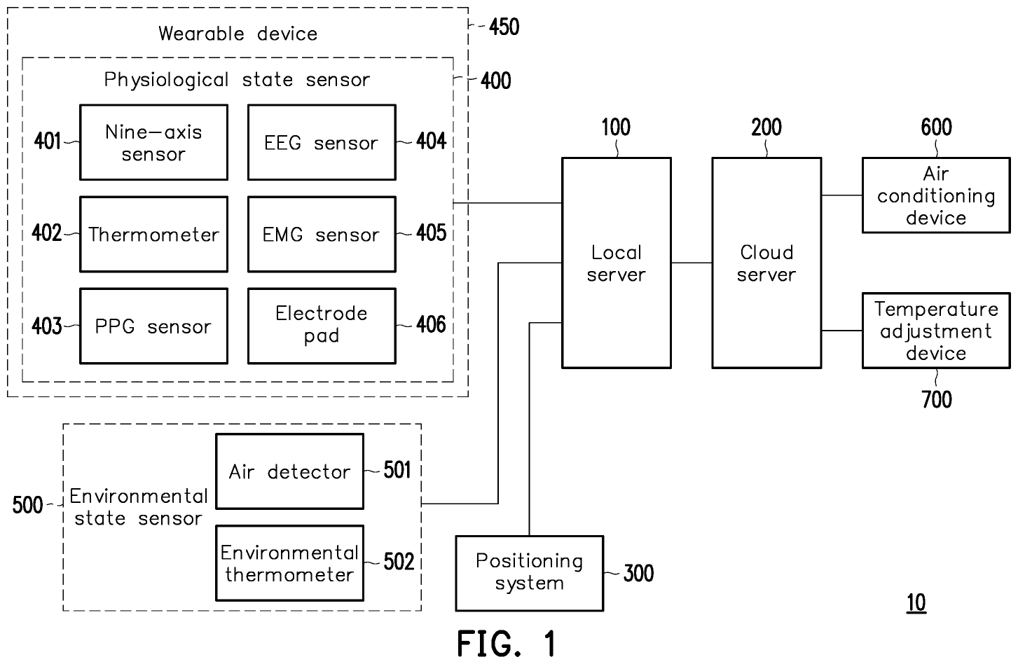

In the invention, brain disease risk or cardiovascular disease risk may be predicted according to some early signs or leading indicators to prompt users to pay attention to their own physiological state. FIG. 1 shows a diagram of a health management system 10 according to an embodiment of the invention. The health management system 10 may monitor the physiological state of a person in a specific space around the clock to collect physiological state information or environmental state information of the person when a major event occurs, wherein the specific space may include indoor space or outdoor space. The health management system 10 may also analyze the collected physiological state information or environmental state information to determine whether to issue an alarm, so as to guide the user to understand the adverse effects of living habits, prevent the occurrence of diseases, or urgently call other personnel to assist those in need.

The health management system 10 may include a local server 100, a cloud server 200, a positioning system 300, a physiological state sensor 400, a wearable device 450, an environmental state sensor 500, an air conditioning device 600, and a temperature adjustment device 700. The local server 100 may be communicatively connected to the cloud server 200, the positioning system 300, the physiological state sensor 400, and the environmental state sensor 500, and may forward the data collected by the positioning system 300, the physiological state sensor 400, or the environmental state sensor 500 to the cloud server 200.

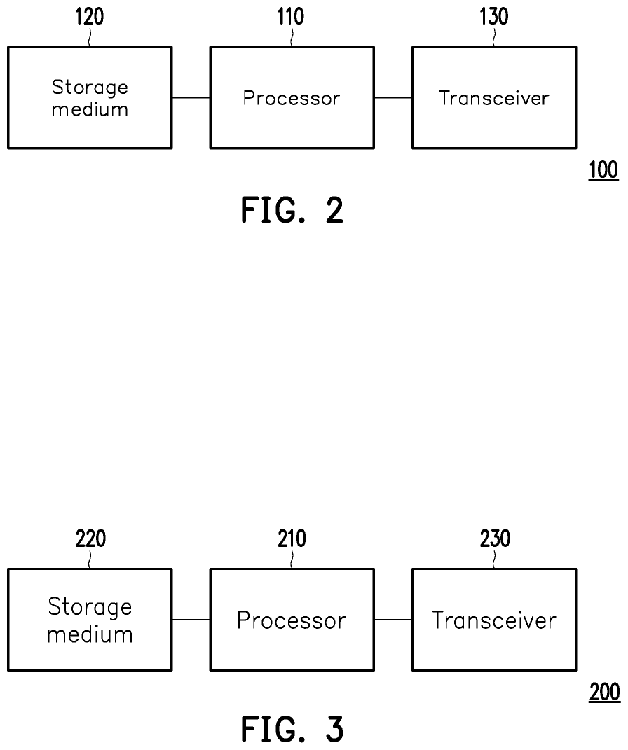

FIG. 2 shows a diagram of the local server 100 according to an embodiment of the invention. The local server 100 is, for example, a gateway or a smart phone, but the invention is not limited thereto. The local server 100 may include a processor 110, a storage medium 120, and a transceiver 130.

The processor 110 is, for example, a central processing unit (CPU), or other programmable general-purpose or special-purpose micro control units (MCU), microprocessors, digital signal processors (DSP), programmable controllers, application-specific integrated circuits (ASIC), graphics processing units (GPU), image signal processors (ISP), image processing units (IPU), arithmetic logic units (ALU), complex programmable logic devices (CPLD), field-programmable gate arrays (FPGA), or other similar elements or a combination of the above elements. The processor 110 may be coupled to the storage medium 120 and the transceiver 130, and access and execute a plurality of modules and various applications stored in the storage medium 120.

The storage medium 120 is, for example, any type of fixed or removable random-access memory (RAM), read-only memory (ROM), flash memory, hard disk drive (HDD), solid state drive (SSD), or similar elements or a combination of the above elements configured to store a plurality of modules or various applications that may be executed by the processor 110.

The transceiver 130 transmits and receives a signal in a wireless or wired manner. The transceiver 130 may also execute operations such as low noise amplification, impedance matching, frequency mixing, up or down frequency conversion, filtering, amplification, and the like. The local server 100 may be communicatively connected to the cloud server 200, the positioning system 300, the physiological state sensor 400, and the environmental state sensor 500 via the transceiver 130.

The cloud server 200 may be communicatively connected to the air conditioning device 600 and the temperature adjustment device 700, and analyze the data from the local server 100 to control the air conditioning device 600 or the temperature adjustment device 700 according to the data. FIG. 3 shows a diagram of the cloud server 200 according to an embodiment of the invention. The cloud server 200 may include a processor 210, a storage medium 220, and a transceiver 230.

The processor 210 is, for example, a central processing unit, or other programmable general-purpose or special-purpose micro-control units, microprocessors, digital signal processors, programmable controllers, special-application integrated circuits, graphics processors, image signal processors, image processing units, arithmetic logic units, complex programmable logic devices, field-programmable logic gate arrays, or other similar elements or a combination of the above elements. The processor 210 may be coupled to the storage medium 220 and the transceiver 230, and access and execute a plurality of modules and various applications stored in the storage medium 220.

The storage medium 220 is, for example, any type of fixed or removable random-access memory (RAM), read-only memory (ROM), flash memory, hard disk drive (HDD), solid state drive (SSD), or similar elements or a combination of the above elements configured to store a plurality of modules or various applications that may be executed by the processor 210.

The transceiver 230 transmits and receives a signal in a wireless or wired manner. The transceiver 130 may also execute operations such as low noise amplification, impedance matching, frequency mixing, up or down frequency conversion, filtering, amplification, and the like. The cloud server 200 may be communicatively connected to the local server 100, the air conditioning device 600, and the temperature adjustment device 700 via the transceiver 230.

In an embodiment, the cloud server 200 may transmit an alarm related to the physiological state information or the environmental state information to an external electronic device via the transceiver 230. For example, the cloud server 200 may receive an access request from a terminal device of the user via the transceiver 230 and send a physiological state report related to the physiological state information to the terminal device to prompt the user whether their physiological state is abnormal. Alternatively, the cloud server 200 may automatically transmit the physiological state report to a preset terminal device after the physiological state report is generated. For another example, the cloud server 200 may send an alarm related to the physiological state information to the terminal devices of medical staff or firefighters via the transceiver 230, so as to prompt the medical staff to help people with abnormal physiological states. For another example, the cloud server 200 may send an alarm related to the environmental state information (for example, the environmental temperature is too high) to the firefighters via the transceiver 230 to prompt the firefighters to put out the fire.

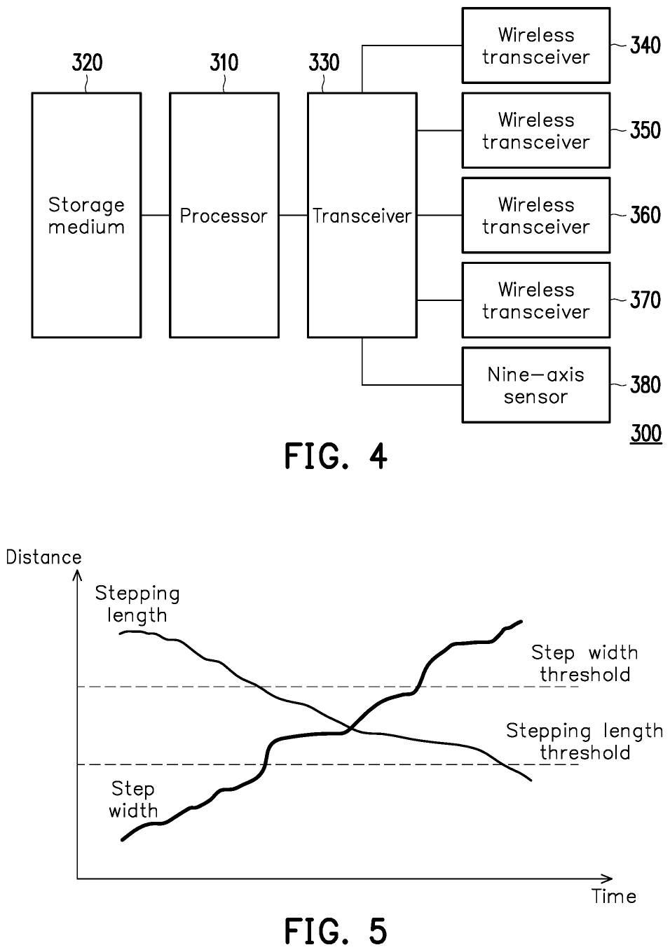

The positioning system 300 is used to measure the position information of a person in a specific space. FIG. 4 shows a diagram of the positioning system 300 according to an embodiment of the invention. The positioning system 300 may include a processor 310, a storage medium 320, a transceiver 330, a wireless transceiver 340, a wireless transceiver 350, a wireless transceiver 360, a wireless transceiver 370, and a nine-axis sensor 380.

The processor 310 is, for example, a central processing unit, or other programmable general-purpose or special-purpose micro-control units, microprocessors, digital signal processors, programmable controllers, special-application integrated circuits, graphics processors, image signal processors, image processing units, arithmetic logic units, complex programmable logic devices, field-programmable logic gate arrays, or other similar elements or a combination of the above elements. The processor 310 may be coupled to the storage medium 320 and the transceiver 330, and access and execute a plurality of modules and various applications stored in the storage medium 320.

The storage medium 320 is, for example, any type of fixed or removable random-access memory (RAM), read-only memory (ROM), flash memory, hard disk drive (HDD), solid state drive (SSD), or similar elements or a combination of the above elements configured to store a plurality of modules or various applications that may be executed by the processor 310.

The transceiver 330 transmits and receives a signal in a wireless or wired manner. The transceiver 330 may also execute operations such as low noise amplification, impedance matching, frequency mixing, up or down frequency conversion, filtering, amplification, and the like. The transceiver 330 may be coupled to the wireless transceiver 340, the wireless transceiver 350, the wireless transceiver 360, the wireless transceiver 370, and the nine-axis sensor 380. In addition, the positioning system 300 may also be communicatively connected to the local server 100 via the transceiver 330.

The wireless transceiver 340, the wireless transceiver 350, the wireless transceiver 360, and the wireless transceiver 370 may have the function of transmitting or receiving a wireless signal. The wireless transceiver 340 may be disposed at the wearable device 450 or at a portable device held by the user (for example, a smart phone). The wireless transceiver 350, the wireless transceiver 360, and the wireless transceiver 370 may be respectively disposed at different positions in a specific space. For example, the wireless transceiver 350, the wireless transceiver 360, and the wireless transceiver 370 may be respectively disposed in a plurality of lamps at different positions on the ceiling, but the invention is not limited thereto.

In an embodiment, the processor 310 of the positioning system 300 may transmit a signal via the wireless transceiver 340, and may receive the signal via the wireless transceiver 350, the wireless transceiver 360, and the wireless transceiver 370 to execute a triangulation positioning measurement, thereby generating a position information corresponding to the wearable device 450. For example, the processor 310 may determine the distance between the wireless transceiver 340 and the wireless transceiver 350 (or the wireless transceiver 360 and the wireless transceiver 370) according to the received signal strength indication (RSSI) of the received signal, so as to execute the triangulation positioning measurement according to the distance. The positioning system may transmit the position information to the local server 100 via the transceiver 330, so that the local server 100 forwards the position information (or “first position information”) to the cloud server 200.

In an embodiment, the processor 310 of the positioning system 300 may transmit a plurality of signals respectively via the wireless transceiver 350, the wireless transceiver 360, and the wireless transceiver 370, and may receive the plurality of signals via the wireless transceiver 340 to execute the triangulation positioning measurement to generate the position information corresponding to the wearable device 450. The positioning system may transmit the position information to the local server 100 via the transceiver 330, so that the local server 100 forwards the position information (or “second position information”) to the cloud server 200. The processor 210 of the cloud server 200 may correct the first position information using the second position information or correct the second position information using the first position information, so as to obtain more accurate position information.

In an embodiment, the storage medium 320 of the positioning system 300 may prestore a magnetic fingerprint corresponding to a specific space. The positioning system 300 may transmit a magnetic signal via the wireless transceiver 350, the wireless transceiver 360, or the wireless transceiver 370, and may receive the magnetic signal via the wireless transceiver 340. The processor 310 of the positioning system 300 may determine the position information corresponding to the wearable device 450 according to the magnetic signal received by the wireless transceiver 340 and the magnetic fingerprint in the specific space.

The position information generated by the positioning system 300 may be used to provide a navigation function. For example, after the cloud server 200 obtains the position information, if a terminal device transmits an access request to the cloud server 200, the processor 210 of the cloud server 200 may transmit the position information to the terminal device via the transceiver 230. Alternatively, the processor 210 first generates a navigation information according to the position information, and then transmits the navigation information to the terminal device via the transceiver 130. In order to facilitate the integration with the map data of other outdoor positioning systems, by continuously providing the navigation function for a person moving between various buildings or outdoor spaces, the position information generated by the positioning system 300 may be presented in the form of geodetic coordinates or latitude and longitude.

In an embodiment, the nine-axis sensor 380 of the positioning system 300 may be disposed at the wearable device 450, wherein the nine-axis sensor 380 may include a gyroscope for measuring angular velocity, an accelerometer for measuring acceleration, and a magnetometer for measuring a magnetic field. The processor 310 of the positioning system 300 may measure a movement information of a person wearing the wearable device 450 via the nine-axis sensor 380, and generate the position information according to the movement information. The movement information may include a current position, a speed, a heading, or a magnetic strength of a specific direction.

Referring to FIG. 1, the physiological state sensor 400 may be disposed on the wearable device 450 to measure the physiological state information of a person wearing the wearable device 450, wherein the wearable device 450 is, for example, a smart bracelet, a head-mounted device, or a neck-mounted device, and the wearable device 450 may include a wireless transceiver for transmitting or receiving a signal. In addition, the physiological state sensor 400 may also be directly worn by a person. The physiological state sensor 400 may include a nine-axis sensor 401, a thermometer 402, a photoplethysmography (PPG) sensor 403, an electroencephalography (EEG) sensor 404, an electromyography (EMG) sensor 405, and an electronic pad 406, wherein the nine-axis sensor 401 and the nine-axis sensor 380 may be the same or different nine-axis sensors.

The type of the sensor disposed on the wearable device 450 may be related to the type of the wearable device 450. For example, if the wearable device 450 is a shoe, the physiological state sensor 400 may include the nine-axis sensor 401. If the wearable device 450 is a smart bracelet, the physiological state sensor 400 may include the nine-axis sensor 401, the thermometer 402, and the PPG sensor 403. If the wearable device 450 is a head-mounted device, the physiological state sensor 400 may include the nine-axis sensor 401, the thermometer 402, the PPG sensor 403, and the EEG sensor 404. If the wearable device 450 is a neck-mounted device, the physiological state sensor 400 may include the nine-axis sensor 401, the thermometer 402, and the PPG sensor 403. The physiological state sensor 400 may measure the physiological state information of the user. The cloud server 200 may evaluate whether the user has potential brain disease risk or cardiovascular disease risk by analyzing long-term accumulated physiological state information, and generate a corresponding physiological state report to warn the user.

If the wearable device 400 is a shoe, the nine-axis sensor 401 may measure a gait information of the user, wherein the gait information may include a time gait parameter and a spatial gait parameter. The time parameter may include walking rate, stride frequency, step time, stride time, swing period, standing period, one-foot support time, or two-foot support time, etc. The gait cycle is the elapsed time between the first floor contact and the second floor contact of the first foot. The walking rate is the distance of movement in the direction of travel per second. Stride frequency is the number of steps taken per minute. The step time is the elapsed time from the first floor contact of the first foot to the first floor contact of the second foot. The stride time is the elapsed time from the first floor contact of the first foot to the second floor contact of the first foot. The swing period is the period during which the first foot leaves the floor in the gait cycle (i.e., stride time) (unit is the percentage of the gait cycle). The standing period is the period during which the first foot touches the floor in the gait cycle (unit is the percentage of the gait cycle). The one-foot support time is the time elapsed from the first foot off the floor to the first foot touching the floor. The two-foot support time is the time between the first foot off the floor and the second foot touching the floor plus the time between the second foot off the floor and the first foot touching the floor.

The spatial gait parameter may include, for example, step width, stepping length, stride length, supporting base, or in/out-toeing. The step width is the distance between the heel of the first foot and the heel of the second foot. The stepping length is the distance in the travel direction from when the heel of the first foot touches the floor to the heel of the second foot touching the floor when walking. The stride length is the distance in the travel direction between the first floor contact and the second floor contact of the heel of the first foot when walking. The supporting base is the distance between the projection of the heel of the footprint of the first foot on the travel path and the projection of the heel of the footprint of the second foot on the travel path. In/out-toeing is the angle between the travel path and the midline of the footprint.

The cloud server 200 may generate a physiological state report including a warning message related to metabolic deterioration risk or brain disease risk according to the gait information. For example, the cloud server 200 may determine the amount of exercise of the user according to the gait information, thereby assessing the metabolism deterioration risk of the user according to the amount of exercise. The cloud server 200 may further determine the blood pressure, blood oxygen, or blood fat of the user according to the metabolism deterioration risk. If the blood pressure, blood oxygen, or blood fat of the user exceeds the standard, the cloud server 200 may generate a physiological state report including the corresponding warning message.

When the brain of a person degenerates, the stepping length of the person is gradually shortened and the step width is gradually increased. Accordingly, the cloud server 200 may determine whether the person is at risk of brain disease according to the stepping length or the step width. FIG. 5 shows a diagram of a gait information according to an embodiment of the invention. The gait information may include stepping length and step width. The cloud server 200 may generate a physiological state report including a warning message related to a brain disease risk in response to the stepping length being less than a stepping length threshold or the step width being greater than a step width threshold.

If the wearable device 450 is a neck-mounted device, the nine-axis sensor 401 may measure the displacement information of the chest cavity of the user. The cloud server 200 may determine the breathing state or sleep quality of the user according to the displacement information, so as to generate a physiological state report including a warning message related to a metabolism deterioration risk according to the displacement information.

Referring to FIG. 1, the thermometer 402 may measure the body temperature of the user. A body temperature that is too high may cause cardiovascular disease. Accordingly, the cloud server 200 may generate a physiological state report including a warning message related to a cardiovascular disease risk in response to the measured body temperature being greater than a body temperature threshold.

The PPG sensor 403 may measure the heart rate variability (HRV), blood oxygen, or blood pressure of the user. In an embodiment, the cloud server 200 may generate a physiological state report including a warning message related to a brain disease risk or a cardiovascular disease risk in response to the measured blood pressure being greater than a blood pressure threshold. In an embodiment, the cloud server 200 generates the physiological state report including a warning message related to a brain disease risk, a cardiovascular disease risk, or a metabolism deterioration risk according to the heart rhythm variability. Specifically, the cloud server 200 may determine the presence of a brain disease risk, a cardiovascular disease risk, or a metabolic deterioration risk via a plurality of indicators calculated based on heart rhythm variability, wherein the plurality of indicators may include standard deviation of NN intervals (SDNN), root mean square successive differences (RMSSD), total power (TP), low-frequency power (LF), high-frequency power (HF), or LF/HF.

The EEG sensor 404 may measure the EEG of the user. The EEG may include a plurality of waves such as Alpha (α) wave, Beta (β) wave, Gamma (γ) wave, Delta (δ) wave, Theta (θ) wave, Lambda (λ) wave, or P300 wave. The cloud server 200 may determine the secretion of dopamine in the brain of the user according to the waveforms of the plurality of waves, and then generate a physiological state report including a warning message related to a brain disease risk according to the determination result.

The EMG sensor 405 may measure the EMG of the user. For example, brain disease may affect the chewing ability of the user, so the EMG of the masticatory muscles of the user may be changed due to brain disease. Therefore, the cloud server 200 may generate a physiological state report including a warning message related to a brain disease risk according to the EMG.

The electrode pad 406 may be attached to the abdominal cavity of the user, and may measure the bowel sound of the user. The cloud server 200 may generate a physiological state report including a warning message related to a metabolic deterioration risk according to the bowel sound.

The environmental state sensor 500, the air conditioning device 600, and the temperature adjustment device 700 may be disposed in a specific space monitored by the health management system 10, wherein the air conditioning device 600 and the temperature adjustment device 700 may be controlled by the processor 210 of the cloud server 200. The environmental state sensor 500 may include an air detector 501 and an environmental thermometer 502. The environmental state sensor 500 may be used to measure an environmental state information. The local server 100 may forward the environmental state information to the cloud server 200. The environmental state information may include the air quality measured by the air detector 501. The cloud server 200 may determine whether to activate the air conditioning device 600 according to the air quality to improve the air quality. The environmental state information may also include an environmental temperature measured by the environmental thermometer 502. The cloud server 200 may determine whether to activate the temperature adjustment device 700 according to the environmental temperature to adjust the environmental temperature to a suitable temperature. In other words, if the air quality or the environmental temperature does not need to be adjusted, the air conditioning device 600 or the temperature adjustment device 700 may be kept in a closed state to save power consumption.

The local server 100 may determine whether to measure the physiological state information of the person using the physiological state sensor 400 or measure the environmental state information using the environmental state sensor 500 according to at least one of a position information, a time information, an environmental temperature, and an air quality. The processor 110 of the local server 100 may transmit the physiological state information or the environmental state information to the cloud server 200 via the transceiver 130, so that the processor 210 of the cloud server 200 generates a physiological state report according to the physiological state information, or determine whether to activate the air conditioning device 600 or the temperature adjustment device 700 according to the environmental state information. Specifically, the storage medium 120 of the local server 100 may pre-store a mapping table related to at least one of a position information, a time information, an environmental temperature, and an air quality. The processor 110 of the local server 100 may determine whether to activate the sensor (the physiological state sensor 400 or the environmental state sensor 500) and the type of sensor to be activated according to the mapping table, and may determine whether to activate the air conditioning device 600 or the temperature adjustment device 700 according to the mapping table.

In an embodiment, the local server 100 may determine the time of a preset position of the person in a specific space according to the position information and the time information, and may measure the physiological state information of the person using the physiological state sensor 400 corresponding to the preset position in response to the time being greater than a time threshold or the time being within a specific time interval. Accordingly, the local server 100 may monitor the physiological state of the person all-weather, and control the physiological state sensor 400 to measure the physiological state information when a major event occurs. When no major event occurs, the physiological state sensor 400 may be kept in a closed state to save power consumption.

TABLE 1

| | | Air quality or | |
|---|---|---|---|
| Position | Time interval or | environmental | Type of device |
| information | time threshold | temperature | activated |
| Bedroom | 23:00 to 06:00 | | EEG sensor 404 |
| Bedroom | 23:00 to 06:00 | 25° C. to 28° C. | Temperature |
| | | | adjustment |
| | | | device 700 |
| Living | 10 minutes | | Environmental |
| room | | | thermometer 502 |
| Bathroom | | >60° C. | Thermometer 402 |
| Bathroom | 30 minutes | | PPG sensor 403 |
| Kitchen | | PM2.5 35 μg/m\hat{ }3 | Air conditioning |
| | | | device 600 |

Taking Table 1 as an example, the mapping table pre-stored in the storage medium 120 of the local server 100 may store the information shown in Table 1. If the local server 100 determines that the person is staying in the bedroom during the time interval 23:00 to 06:00, the local server 100 may determine to activate the EEG sensor 404 to measure the EEG of the person, so as to determine the sleep quality of the person according to the EEG. If the environmental temperature is not between 25° C. and 28° C., the local server 100 may determine to activate the temperature adjustment device 700 to adjust the environmental temperature to between 25° C. and 28° C. If the local server 100 determines that the person stays in the living room for more than 10 minutes, the local server 100 may determine to activate the environmental thermometer 502 to measure the environmental temperature of the living room. The local server 100 may further determine whether to activate the temperature adjustment device 700 according to the environmental temperature. If the local server 100 determines that the person is located in the bathroom and the environmental temperature of the bathroom is greater than 60° C., the local server 100 may activate the thermometer 402 to measure the body temperature of the person. The local server 100 may further determine whether the body temperature of the person is too high according to the temperature. If the local server 100 determines that the person stays in the bathroom for more than 30 minutes, the local server 100 may activate the PPG sensor 403 to measure the blood pressure of the person. The local server 100 may further determine whether the person may be unconscious due to high blood pressure according to the blood pressure. If the local server 100 determines that the person is in the kitchen and the PM2.5 of the kitchen is greater than 35 μg/m\hat{ }3, the local server 100 may activate the air conditioning device 600 to filter the air and improve the air quality.

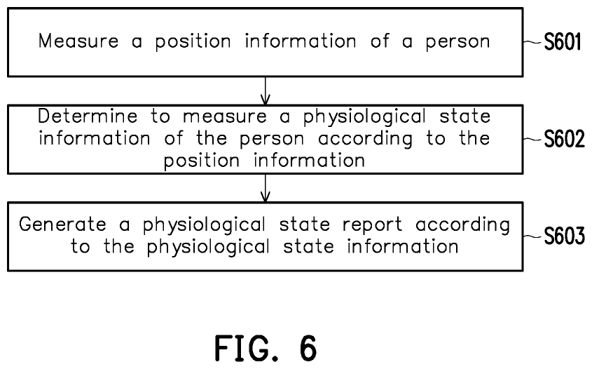

FIG. 6 shows a flowchart of a health management method according to an embodiment of the invention, wherein the health management method may be implemented by the health management system 10 shown in FIG. 1. In step S601, a position information of a person is measured. In step S602, it is determined to measure a physiological state information of the person according to the position information. In step S603, a physiological state report is generated according to the physiological state information.

Based on the above, the health management system of the invention may include various physiological state sensors and environmental state sensors. The health management system may integrate these sensors, and monitor the physiological state and the environmental state of the person in a specific space via the sensors to collect big data. The physiological state sensor may be disposed on the wearable device to accurately measure the physiological state information of the person. The health management system may continuously monitor the life trajectory of a person via the positioning system, and begin to measure and analyze the physiological state information of the person when a major event occurs. The health management system may predict the physiological state abnormalities that may occur in the person by analyzing the physiological state information and send out a real-time alarm to avoid missing the critical rescue time. Therefore, in the invention, the user may be guided to understand the adverse effects of living habits without infringing on the privacy of the user, thereby preventing the occurrence of diseases (such as cardiovascular disease risk or brain disease risk).

What is claimed is:

1. A health management system suitable for monitoring a physiological state of a person in a specific space, comprising:
a physiological state sensor;
a positioning system measuring a position information of the person;
a local server communicatively connected to the positioning system of the physiological state sensor, wherein the local server determines to measure a physiological state information of the person using the physiological state sensor according to the position information; and
a cloud server communicatively connected to the local server, wherein the cloud server generates a physiological state report according to the physiological state information.

2. The health management system of claim 1, further comprising:
a wearable device, wherein the physiological state sensor is disposed at the wearable device to measure the physiological state information of the person wearing the wearable device.

3. The health management system of claim 2, wherein the wearable device comprises a shoe, the physiological state sensor comprises a nine-axis sensor, and the physiological state information comprises a gait information.

4. The health management system of claim 2, wherein the physiological state sensor comprises a photoplethysmography sensor, and the physiological state information comprises a heart rhythm variability and a blood pressure.

5. The health management system of claim 2, wherein the physiological state sensor comprises an electroencephalography sensor, and the physiological state information comprises an electroencephalogram.

6. The health management system of claim 2, wherein the physiological state sensor comprises a thermometer, and the physiological state information comprises a body temperature.

7. The health management system of claim 2, wherein the physiological state sensor comprises an electromyography sensor, and the physiological state information comprises an electromyogram.

8. The health management system of claim 2, wherein the physiological state sensor comprises an electrode pad, and the physiological state information comprises a bowel sound.

9. The health management system of claim 2, wherein the wearable device comprises a neck-mounted device, the physiological state sensor comprises a nine-axis sensor, and the physiological state information comprises a displacement information.

10. The health management system of claim 3, wherein the cloud server generates the physiological state report comprising a warning message related to a brain disease risk or a metabolism deterioration risk according to the gait information.

11. The health management system of claim 10, wherein the gait information comprises a stepping length and a step width, and the cloud server generates the physiological state report comprising the warning message related to the brain disease risk in response to the stepping length being less than a stepping length threshold or the step width being greater than a step width threshold.

12. The health management system of claim 4, wherein the cloud server generates the physiological state report comprising a warning message related to a brain disease risk or a cardiovascular disease risk in response to the blood pressure being greater than a blood pressure threshold.

13. The health management system of claim 4, wherein the cloud server generates the physiological state report comprising a warning message related to a brain disease risk, a cardiovascular disease risk, or a metabolic deterioration risk according to the heart rhythm variability.

14. The health management system of claim 5, wherein the cloud server generates the physiological state report comprising a warning message related to a brain disease risk according to the electroencephalogram.

15. The health management system of claim 6, wherein the cloud server generates the physiological state report comprising a warning message related to a cardiovascular disease risk in response to the body temperature being greater than a body temperature threshold.

16. The health management system of claim 7, wherein the cloud server generates the physiological state report comprising a warning message related to a brain disease risk according to the electromyogram.

17. The health management system of claim 8, wherein the cloud server generates the physiological state report comprising a warning message related to a metabolism deterioration risk according to the bowel sound.

18. The health management system of claim 9, wherein the cloud server generates the physiological state report comprising a warning message related to a metabolism deterioration risk according to the displacement information.

19. The health management system of claim 4, wherein the wearable device comprises a smart bracelet, a head-mounted device, or a neck-mounted device.

20. The health management system of claim 5, wherein the wearable device comprises a head-mounted device.

21. The health management system of claim 6, wherein the wearable device comprises a smart bracelet, a head-mounted device, or a neck-mounted device.

22. The health management system of claim 1, further comprising:
an environmental state sensor communicatively connected to the local server, wherein the local server measures an environmental state information using the environmental state sensor.

23. The health management system of claim 22, further comprising:
an air conditioning device communicatively connected to the cloud server, wherein the environmental state sensor comprises an air detector, the environmental state information comprises an air quality, and the cloud server activates the air conditioning device according to the air quality.

24. The health management system of claim 22, further comprising:
a temperature adjustment device communicatively connected to the cloud server, wherein the environmental state sensor comprises an environmental thermometer, the environmental state information comprises an environmental temperature, and the cloud server activates the temperature adjustment device according to the environmental temperature.

25. The health management system of claim 1, wherein the local server further determines to measure the physiological state information of the person using the physiological state sensor according to at least one of a time information, an environmental temperature, and an air quality.

26. The health management system of claim 25, wherein the local server determines a time that the person stays at a preset position in the specific space according to the position information and the time information, and measures the physiological state information of the person using the physiological state sensor corresponding to the preset position in response to the time being greater than a time threshold.

27. The health management system of claim 1, further comprising a wearable device, wherein the positioning system comprises a first wireless transceiver, a second wireless transceiver, a third wireless transceiver, and a fourth wireless transceiver, wherein
the first wireless transceiver is disposed at the wearable device; and
the second wireless transceiver, the third wireless transceiver, and the fourth wireless transceiver are respectively disposed at different positions in the specific space.

28. The health management system of claim 27, wherein the positioning system transmits a signal via the first wireless transceiver, and receives the signal via the second wireless transceiver, the third wireless transceiver, and the fourth wireless transceiver to execute a triangulation positioning measurement to generate the position information.

29. The health management system of claim 27, wherein the positioning system transmits a second signal via the second wireless transceiver, transmits a third signal via the third wireless transceiver, transmits a fourth signal via the fourth wireless transceiver, and receives the second signal, the third signal, and the fourth signal via the first wireless transceiver to execute a triangulation positioning measurement to generate the position information.

30. The health management system of claim 27, wherein the positioning system prestores a magnetic fingerprint corresponding to the specific space, the positioning system radiates a magnetic signal via the second wireless transceiver, receives the magnetic signal via the first wireless transceiver, and generates the position information according to the magnetic signal and the magnetic fingerprint received by the first wireless transceiver.

31. The health management system of claim 1, further comprising a wearable device, wherein the positioning system comprises a nine-axis sensor disposed on the wearable device, wherein
the positioning system measures a movement information of the person wearing the wearable device via the nine-axis sensor, and generates the position information according to the movement information.

32. The health management system of claim 31, wherein the movement information comprises at least one of the following:
a current position, a speed, a heading, and a magnetic strength of a specific direction.

33. The health management system of claim 1, wherein the cloud server transmits the physiological state report to a terminal device sending an access request in response to receiving the access request.

34. A health management method suitable for monitoring a physiological state of a person in a specific space, comprising:
measuring a position information of the person;
determining to measure a physiological state information of the person according to the position information; and
generating a physiological state report according to the physiological state information.
